(19) United States
(12) Patent Application Publication
(10) Pub. No.: US 2026/0259185 A1
(43) Pub. Date: Sep. 3, 2026

(54) COMPOSITIONS AND ASSAYS FOR OPIOID DETECTION

(71) Applicant: Trustees of Tufts College, Medford, MA (US)

(72) Inventors: Charles R. Mace, Winchester, MA (US); Andrea C. Mora, Medford, MA (US)

(73) Assignee: Trustees of Tufts College, Medford, MA (US)

(21) Appl. No.: 18/878,337

(22) PCT Filed: Jun. 23, 2023
(86) PCT No.: PCT/US2023/026099

§ 371 Date: Dec. 23, 2024

Related U.S. Application Data
(60) Provisional application No. 63/355,453, filed on Jun. 24, 2022.

(51) Int. Cl. G01N 31/22; G01N 21/78

(52) U.S. Cl. CPC G01N 31/22; G01N 21/78

(57) ABSTRACT

Compositions, kits, and methods for the detection of target analytes are generally provided. In some embodiments, the compositions, kits, and methods described herein comprise a fluid in which a detection agent, an indicator, and a trapping agent are dissolved, suspended, and/or dispersed and allow for safe and/or facile detection of target analytes, including controlled substances such as opioids.

### RELATED APPLICATIONS

This application claims priority under 35 U.S.C. § 119 (e) to U.S. Provisional Application No. 63/355,453, filed Jun. 24, 2022, and entitled “Compositions and Assays for Opioid Detection,” which is incorporated herein by reference in its entirety.

### GOVERNMENT SPONSORSHIP

This invention was made with government support under grants W911QY19P0128 and W911SR21C0019 awarded by the Department of Defense. The government has certain rights in the invention.

### TECHNICAL FIELD

Compositions, kits, and methods for the detection of target analytes are generally described.

### BACKGROUND

Chemically specific detection of analytes is an important problem. Detection of analytes that are controlled substances can, in some instances, present a number of challenges for field detection. For example, common problems include low assay specificity, assay reagent toxicity, and the need for close physical proximity of an assay-performer to an unknown and potentially dangerous substance.

### SUMMARY

Compositions, kits, and methods for the detection of target analytes are generally described. The subject matter of the present disclosure involves, in some cases, interrelated products, alternative solutions to a particular problem, and/or a plurality of different uses of one or more systems and/or articles.

In one aspect, a composition for detecting the presence of a target analyte is provided. The composition for detecting the presence of a target analyte comprises a fluid in which a detection agent, an indicator, and a trapping agent are dissolved, suspended, and/or dispersed. At least a portion of the detection agent is associated with the trapping agent. Interaction of the detection agent with the indicator in the presence of the fluid yields a detectable signal. The detectable signal is absent, has an intensity that is not detectable by eye, and/or has an intensity that is within the range of the noise associated with a signal having zero intensity. The target analyte has a higher affinity for the trapping agent than the detection agent in the presence of the fluid.

In another aspect, a kit for detecting the presence of a target analyte is provided. The kit for detecting the presence of a target analyte comprises a first fluid in which a detection agent and a trapping agent are dissolved, suspended, and/or dispersed. The kit for detecting the presence of the target analyte further comprises a second fluid in which an indicator is dissolved, suspended, and/or dispersed. At least a portion of the detection agent is associated with the trapping agent. Interaction of the detection agent with the indicator in the presence of the first fluid and the second fluid yields a detectable signal. The target analyte has a higher affinity for the trapping agent than the detection agent in the presence of the first fluid and the second fluid.

In another aspect, a method of detecting a target analyte is provided. The method of detecting the target analyte comprises exposing a substance to a fluid and/or a plurality of fluids, detecting the presence or absence of a detectable signal, and determining whether a target analyte is present in the substance based on the presence or absence of the detectable signal. Prior to the exposure of the substance to the fluid and/or the plurality of fluids, a detection agent and a trapping agent are dissolved, suspended, and/or dispersed in the fluid and/or in a first fluid in the plurality of fluids. Prior to the exposure of the substance to the fluid and/or the plurality of fluids, at least a portion of the detection agent in the fluid and/or the first fluid is associated with the trapping agent. Prior to the exposure of the substance to the fluid and/or the plurality of fluids, an indicator is dissolved, suspended, and/or dispersed in the fluid and/or in a second fluid in the plurality of fluids. In the absence of the target analyte, the detectable signal is absent, has an intensity that is not detectable by eye, and/or has an intensity that is within the range of the noise associated with a signal having zero intensity. The target analyte has a higher affinity for the trapping agent than the detection agent in the presence of the fluid and/or the plurality of fluids. The detectable signal arises due to interaction of the detection agent with the indicator in the presence of the fluid and/or the plurality of fluids.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1A presents a schematic illustration of a detection agent and a trapping agent, according to some embodiments;

FIG. 1B presents a schematic illustration of a detection agent and a trapping agent in the presence of an indicator, according to some embodiments;

FIG. 2A presents a schematic illustration of displacement of a detection agent from a trapping agent by a target analyte, according to some embodiments;

FIG. 2B presents a schematic illustration of signal generation resulting from an interaction between a detection agent and an indicator, according to some embodiments;

FIG. 3 presents an exemplary schematic illustration of an interaction between a detection agent, an indicator, and an auxiliary agent, according to some embodiments;

FIG. 4 presents a schematic illustration of detection of a target analyte performed according to a method described herein, according to some embodiments;

FIG. 5 is a chart showing absorption intensity as a function of time for two solutions, in accordance with some embodiments;

FIG. 6 presents photographs of an exemplary composition sprayed onto a control surface lacking a target analyte at various timepoints, according to some embodiments;

FIG. 7 presents photographs of an exemplary composition, sprayed onto a control surface on which a target analyte is disposed at various timepoints, according to some embodiments;

FIG. 8 schematically depicts reactions that occur upon the formation of solutions comprising CB[7] and FCA and their subsequent exposure to fentanyl in the presence of TMB;

FIG. 9 is a chart showing the percent inhibition of the interaction of FCA with TMB as a function of the CB[7] concentration, in accordance with some embodiments;

FIG. 10 is a chart showing the absorbance at 653 nm as a function of time for aqueous solutions for detecting AM in the presence of various concentrations of AM, in accordance with some embodiments;

FIG. 11 shows photographs of various test and displacement solutions at various time points, in accordance with some embodiments;

FIG. 12 shows photographs of solutions in the presence and absence of fentanyl, in accordance with some embodiments; and

FIG. 13 shows selected detection agents and indicators and associated experimental data, in accordance with some embodiments.

### DETAILED DESCRIPTION

Compositions, kits, and methods for the detection of target analytes are generally provided. In some embodiments, the compositions, kits, and methods described herein allow for safe and/or facile detection of target analytes, including controlled substances such as opioids.

In some embodiments, a target analyte is detected upon exposure to one or more fluids and/or a composition and/or kit is provided that allows for such target analyte detection. The use of a fluid or fluids in target analyte detection may provide a number of advantages. For example, such fluid or fluids may be exposed to a substance possibly containing the target analyte from a distance, reducing the risk of exposure to the target analyte of the individual performing the detection technique. Reduced risk of exposure may be particularly advantageous in the context of assaying an unknown substance for dangerous analytes such as opioids, since opioids may be hazardous in relatively small quantities.

In some embodiments, a target analyte is detected based on the presence or absence of a detectable signal that arises due to one or more interactions that occur in the presence of one or more fluids. Similarly, a composition and/or kit may be provided that allows for such target analyte detection. Advantageously, species present in such fluids may be relatively mobile, and so may be capable of interacting on a time scale that is relatively small. This may result in the generation of a detectable signal on a time scale that is also relatively small.

Detectable signals that assist with detection of target analytes may arise due to the interaction of the target analyte with one or more species present in the fluid or fluids in a manner that is indirect. In other words, in some embodiments, interaction of the target analyte with one or more species present in the fluid or fluids causes the detectable signal to be present or absent, but does not participate directly in signal generation. As an example, a target analyte may displace a detection agent from association with a trapping agent, and interaction of the detection agent with an indicator may result in the production of a species that emits a detectable signal. In situations where the trapping agent prevents or reduces the rate at which the detection agent associates with an indicator, a higher affinity of the trapping agent for the target analyte in comparison to the detection agent may be exploited to determine the presence of the target analyte in the fluid.

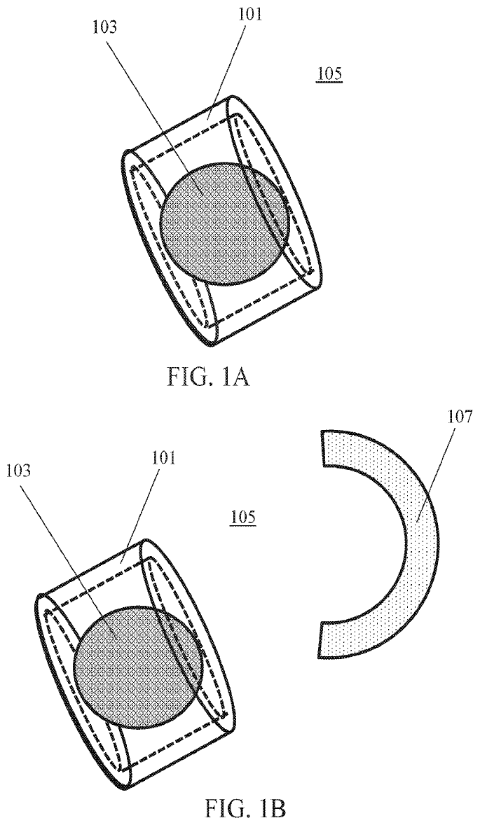

As described above, in some embodiments, a target analyte is detected upon exposure to a fluid in which a trapping agent and a detection agent are each dissolved, suspended, and/or dispersed. Prior to exposure of this fluid to the target analyte, the detection agent may be associated with the trapping agent. For example, FIG. 1A provides a schematic perspective illustration of a trapping agent 101 associated with a detection agent 103 in a fluid 105. A trapping agent may be associated with a detection agent such that the detection agent has a reduced ability to interact with other species, such as indicators. For example, the trapping agent 101 shown in FIG. 1A limits the ability of the detection agent 103 to interact with other chemical species by partially enclosing detection agent 103 and by chemically interacting with it.

Reducing the ability of a detection agent to interact with other species in the context of an assay may be desirable because it may prevent, reduce, and/or delay signal generation. For example, FIG. 1B provides a schematic illustration of an indicator 107, shown in a fluid 105 containing a detection agent 103 and a trapping agent 101. In some instances, the absence of the trapping agent 101 would permit an interaction between the detection agent 103 and the indicator 107, which could result in the production of a detectable signal. However, the presence of the trapping agent 101, and its association with the detection agent 103, reduces, prevents, and/or delays signal generation.

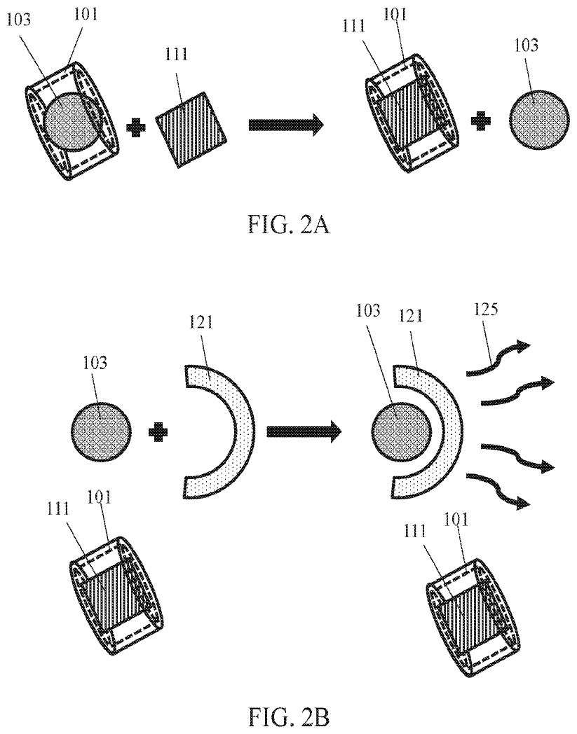

In some embodiments, exposure of a fluid in which a detection agent is associated with a trapping agent to a target analyte causes displacement of the detection agent from the trapping agent by the target analyte. FIG. 2A schematically illustrates one example of such a displacement process. As shown in FIG. 2A, a target analyte 111 may displace a detection agent 103 from a trapping agent 101. Displacement of detection agent 103 may occur regardless of the affinities of target analyte 111 and detection agent 103 for trapping agent 101. However, in some embodiments, it may be advantageous for the trapping agent 101 to have a higher affinity for the target analyte 111 than for the detection agent 103, such that displacement of the detection agent 103 by the target analyte 111 is thermodynamically favored. This may result in a higher degree of displacement of the detection agent 103 by the target analyte 111 and/or more rapid displacement of the detection agent 103 by the target analyte 111, the former of which may result in the production of a signal having higher intensity and the latter of which may result in the production of a signal more rapidly.

The displacement of a detection agent may leave the detection agent free to interact with an indicator. For example, FIG. 2B presents a schematic illustration of a target analyte 111, a trapping agent 101, and a detection agent 103 in the presence of an indicator 121. As shown in FIG. 2B, the detection agent 103 may interact with the indicator 121 to produce a detectable signal 125. Thus, in some embodiments, the mutual presence of an indicator, a target analyte, a detection agent, and a trapping agent in a single fluid or a single mixture of fluids will produce a detectable signal. In some such embodiments, the mutual presence of an indicator, a detection agent, and a trapping agent, in the absence of a target analyte, will not produce a detectable signal. The selective signal generation in the presence of a target analyte may occur because in the absence of the target analyte, the trapping agent may inhibit and/or prevent the interaction between the detection agent and the indicator.

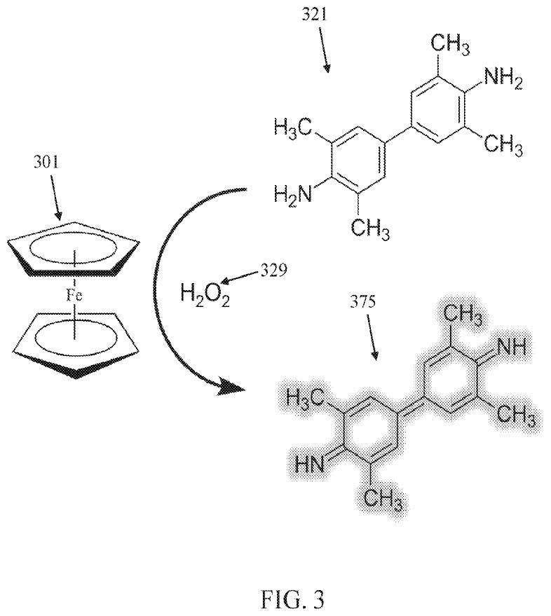

FIG. 3 illustrates one example of an interaction between a detection agent 301 and an indicator 321. In this example, the detection agent 301 (a ferrocene) interacts with the indicator 321 (tetramethylbenzidine) along with an auxiliary agent 329 (hydrogen peroxide). This interaction comprises the oxidation of indicator 321 to form an oxidized indicator 375 (oxidized tetramethylbenzidine). The oxidized indicator may emit a detectable signal.

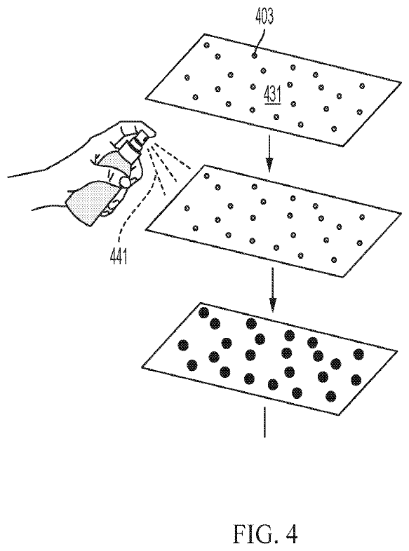

Some embodiments relate to methods for detecting target analytes. Some such methods may make use of one or more of the processes described above with respect to FIGS. 1-3. Additionally, FIG. 4 presents a schematic illustration of one method of detecting a target analyte by exposing the target analyte to a fluid in which a detection agent, an indicator, and a trapping agent are dissolved, suspended, and/or dispersed. In FIG. 4, a substance 403 is shown in the form of a powder on a surface 431. A fluid 441 comprising an indicator, a detection agent, a trapping agent, and an optional auxiliary agent is sprayed onto the surface 431. After fluid 441 contacts the substance 403, it experiences a color change, as shown, demonstrating the presence of a target analyte.

In some embodiments, a composition and/or a kit is provided that comprises one or more fluids in which one or more processes shown in FIGS. 1-3 may occur and/or that are suitable for performing the method shown in FIG. 4. Such compositions and/or kits may comprise one or more fluids in which the indicators, trapping agents, and detection agents shown above are dissolved, suspended, and/or dispersed. In such embodiments, any or all components of a fluid (e.g., a detection agent, a trapping agent, an indicator, and optionally an analyte and/or an auxiliary agent as discussed below) may be independently dissolved, suspended, or dispersed within one or more of the fluid(s) present in such compositions and kits.

In some embodiments, an indicator, a detection agent, and a trapping agent are present in a single fluid to which a substance may be exposed (e.g., by spraying the fluid onto the substance). It is also possible for multiple fluids to be mixed to produce a single fluid that comprises a detection agent, a trapping agent, and an indicator. For example, a substance may be exposed to a first fluid comprising a detection agent and a trapping agent and a second fluid comprising an indicator. In some embodiments, two separate fluids may be exposed to a substance such that they mix in the substance's presence, allowing determination of the presence of a target analyte within the substance.

Some fluids described herein are aqueous fluids and/or aqueous solutions. For example, a composition and/or a kit may comprise an aqueous fluid in which a trapping agent, a detection agent, and/or an indicator are each dissolved, suspended, and/or dispersed. In some embodiments, a fluid comprises an organic solvent comprising dimethyl sulfoxide (DMSO), dimethylformamide, and/or ethanol.

As discussed above, in some embodiments, a fluid comprises a trapping agent. A trapping agent may be configured to associate with a detection agent and/or a target analyte. In some embodiments, the association between a trapping agent and a detection agent is reversible. For example, a detection agent and a trapping agent may reach an equilibrium in a fluid that is characterized by an association constant, Kₐ. The equilibrium may be a dynamic equilibrium such that the detection agent and the trapping agent can reversibly associate and de-associate with each other at equilibrium. In such embodiments, a reduction in the concentration of free trapping agent (e.g., via the association of the trapping agent with a target analyte) may cause an increase in the concentration of free detection agent.

In fluids comprising both a trapping agent and a detection agent and lacking a target analyte, it is possible for a relatively high amount of the detection agent to be associated with the trapping agent and/or for a relatively low amount of the detection agent to be dissociated from the trapping agent. For example, a detection agent and a trapping agent with an appropriate association constant (as described above) may be added to a fluid lacking a target analyte in appropriate concentrations to ensure that most of the detection agent is associated with the trapping agent and the fluid. As a more specific example, a trapping agent may be present in a fluid in a higher molar concentration than a detection agent, which may reduce or eliminate the presence of excess detection agent (i.e., detection agent not associated with the trapping agent) in the fluid.

In some embodiments, a fluid comprises a trapping agent and a detection agent that are allowed to associate with one another prior to exposure to a substance possibly comprising a target analyte. Inclusion of a trapping agent and a detection agent in the same fluid prior to exposure to such a substance may prevent off-target signal generation that could otherwise result from an interaction between the detection agent and an indicator in the presence of the target analyte before the detection agent has sufficient time to associate with the trapping agent. Accordingly, pre-association of a trapping agent and a detection agent may thus improve the accuracy of analyte detection. A trapping agent and a detection agent before exposure to a substance possibly comprising an analyte may remain in the same fluid for the duration of performance of an assay.

In some embodiments, a fluid comprising a detection agent and a trapping agent is exposed to a target analyte before an indicator is introduced into the fluid. Exposure to a target analyte may cause the displacement of a detection agent in a fluid, such that the detection agent is primed to interact with an indicator that is subsequently added to the fluid. A solution comprising a trapping agent and a detection agent may alternatively include an indicator prior to exposure to a target analyte or unknown substance. In some such embodiments, the association between a trapping agent and a detection agent inhibits or prevents the interaction between an indicator and the detection agent, thereby limiting or preventing signal generation that is not associated with the presence of a target analyte.

Trapping agents can associate with other species (e.g., detection agents, target analytes) in a variety of suitable manners. When a trapping agent is capable of associating with two or more species, it may be capable of associating with these species in the same manner or may be capable of associating with different species in different manners. As described above, in some embodiments, association of a trapping agent with one species (e.g., a target analyte) may prevent it from associating with or reduce the rate and/or extent of its association with another species (e.g., a detection agent). In some embodiments, a trapping agent can associate with another species by forming a non-covalent bond therewith. Non-limiting examples of such bonds include hydrogen bonds, aromatic stacking interactions, ionic bonds, ion-dipole interactions, and dipole-dipole interactions. It is also possible for a trapping agent to be capable of associating with another species via a guest-host interaction (e.g., where the trapping agent is the host and the detection agent or target analyte is the guest).

Trapping agents may have a variety of suitable structures. In some embodiments, a trapping agent is or comprises a macrocycle. Macrocycles may have a number of advantageous properties for use in trapping agents. For example, without wishing to be bound by any particular theory, a macrocycle may partially enclose a detection agent capable of fitting within the macrocycle, sterically limiting the ability of the detection agent to interact with an indicator. In some embodiments, a trapping agent is a cucurbituril or a derivative thereof. For example, a trapping agent may be a cucurbit[n]uril where n is 5, 6, 7, 8, 10, or 14 or may be a derivative thereof. In some embodiments, a trapping agent is cucurbit[7]uril. A trapping agent may have one of the chemical structures:

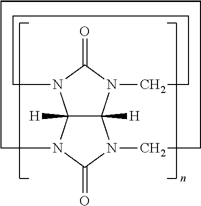

wherein n is 5, 6, 7, 8, or 10.

Cucurbit[n]uril compounds such as cucurbit[n]uril may be particularly well suited for the detection of some analytes, such as opioids and amphetamines. Other compounds, such as calabadions, cyclodextrins, calixarenes, pillarenes, or derivatives thereof may also be used as trapping agents. For example, sulfocalix[4] arene may be a detection agent according to some embodiments.

A trapping agent described herein may have any of a variety of appropriate affinities for a detection agent. The affinity between a trapping agent and a detection agent may be parametrized by an association constant, Kₐ, such that higher affinities are associated with higher association constants. In some embodiments, an association constant, Kₐ, between a detection agent and a trapping agent is greater than or equal to 1×10⁴, greater than or equal to 2×10⁴, greater than or equal to 3×10⁴, greater than or equal to 4×10⁴, greater than or equal to 5×10⁴, greater than or equal to 6×10⁴, greater than or equal to 7×10⁴, greater than or equal to 8×10⁴, greater than or equal to 9×10⁴, greater than or equal to 1×10⁵, greater than or equal to 2×10⁵, greater than or equal to 3×10⁵, greater than or equal to 4×10⁵, greater than or equal to 5×10⁵, greater than or equal to 6×10⁵, greater than or equal to 7×10⁵, greater than or equal to 8×10⁵, greater than or equal to 9×10⁵, greater than or equal to 1×10⁶, greater than or equal to 1.1×10⁶, or greater than or equal to 1.2×10⁶. In some embodiments a Kₐ between a detection agent and a trapping agent is less than or equal to 1.3×10⁶, less than or equal to 1.2×10⁶, less than or equal to 1.1×10⁶, less than or equal to 1×10⁶, less than or equal to 9×10⁵, less than or equal to 8×10⁵, less than or equal to 7×10⁵, less than or equal to 6×10⁵, less than or equal to 5×10⁵, less than or equal to 4×10⁵, less than or equal to 3×10⁵, less than or equal to 2×10⁵, less than or equal to 1×10⁵, less than or equal to 9×10⁴, less than or equal to 8×10⁴, less than or equal to 7×10⁴, less than or equal to 6×10⁴, less than or equal to 5×10⁴, less than or equal to 4×10⁴, less than or equal to 3×10⁴, or less than or equal to 2×10⁴. Combinations of these ranges are also possible (e.g., greater than or equal to 1×10⁴ and less than or equal to 1.3×10⁶, or greater than or equal to 1×10⁵ and less than or equal to 1×10⁶). Other ranges are also possible. In some embodiments, a Kₐ between a detection agent and a trapping agent is high enough that when the detection agent and the trapping agent are present in identical molar concentrations, a majority of the detection agent is associated with the trapping agent.

In some embodiments, a trapping agent has a higher affinity for a target analyte than for a detection agent in the presence of a fluid. Without wishing to be bound by theory, trapping agents having higher affinities for a target analyte than for a detection agent may be more amenable to displacement of the detection agent from the trapping agent by the target analyte. A trapping agent described herein may have any of a variety of appropriate affinities for an analyte. In some embodiments, an association constant, Kₐ, between an analyte and a trapping agent is greater than or equal to 2×10⁶, greater than or equal to 5×10⁶, greater than or equal to 1×10⁷, greater than or equal to 2×10⁷, greater than or equal to 5×10⁷, greater than or equal to 1×10⁸, greater than or equal to 2×10⁸, or greater than or equal to 5×10⁸. In some embodiments a Kₐ between an analyte and a trapping agent is less than or equal to 1×10⁹, less than or equal to 5×10⁸, less than or equal to 2×10⁸, less than or equal to 1×10⁸, less than or equal to 5×10⁷, less than or equal to 2×10⁷, less than or equal to 1×10⁷, or less than or equal to 5×10⁶. Combinations of these ranges are also possible (e.g., greater than or equal to 2×10⁶ and less than or equal to 1×10⁹). Other ranges are also possible.

It should be understood that although it may be advantageous for a trapping agent have a higher affinity for a target analyte than for a detection agent, this is not required. For example, a trapping agent with a higher affinity for a detection agent than for a target analyte may still release at least some of the detection agent in the presence of the target analyte in a high molar excess.

Nonetheless, it may be advantageous for a trapping agent to have a relatively high ratio of the Kₐ between a trapping agent and a target analyte to the Kₐ between the trapping agent and a detection agent. In some embodiments, a trapping agent has a ratio of the Kₐ between the trapping agent and a target analyte to the Kₐ between the trapping agent and a detection agent of greater than or equal to 10, greater than or equal to 20, greater than or equal to 50, greater than or equal to 100, greater than or equal to 200, greater than or equal to 500, greater than or equal to 1000, greater than or equal to 2000, or greater than or equal to 5000. In some embodiments, a trapping agent has a ratio of the Kₐ between the trapping agent and a target analyte to the Kₐ between the trapping agent and a detection agent of less than or equal to 10000, less than or equal to 5000, less than or equal to 2000, less than or equal to 1000, less than or equal to 500, less than or equal to 200, less than or equal to 100, less than or equal to 50, or less than or equal to 20. Combinations of these ranges are also possible (e.g., greater than or equal to 10 and less than or equal to 10000). Other ranges are also possible.

A fluid herein may comprise a trapping agent in any of a variety of suitable concentrations. In some embodiments, a fluid comprises a trapping agent at a concentration of greater than or equal to 0.1 mM, greater than or equal to 0.2 mM, greater than or equal to 0.5 mM, greater than or equal to 1 mM, greater than or equal to 2 mM, greater than or equal to 5 mM, greater than or equal to 10 mM, greater than or equal to 20 mM, or greater than or equal to 50 mM. In some embodiments, a fluid comprises a trapping agent at a concentration of less than or equal to 100 mM, less than or equal to 50 mM, less than or equal to 20 mM, less than or equal to 10 mM, less than or equal to 5 mM, less than or equal to 2 mM, less than or equal to 1 mM, less than or equal to 0.5 mM, or less than or equal to 0.2 mM. Combinations of these ranges are also possible (e.g., greater than or equal to 0.1 mM and less than or equal to 100 mM, greater than or equal to 5 mM and less than or equal to 50 mM, or greater than or equal to 10 mM and less than or equal to 20 mM). Other ranges are also possible. In some embodiments, a fluid does not comprise a trapping agent (e.g., a kit may comprise one fluid comprising a trapping agent and another lacking the trapping agent).

As described above, in some embodiments, a detection agent is suspended, dispersed, or dissolved in a fluid described herein. Generally, a detection agent can associate with a trapping agent (as described elsewhere herein) and/or interact with an indicator. Without wishing to be bound by theory, a detection agent may interact with an indicator in any of a variety of appropriate ways to produce a detectable signal. For example, a detection agent may chemically react with an indicator to produce a detectable signal. It is also possible for an interaction between a detection agent and an indicator to be something other than a chemical reaction.

The detection agent may be a redox reagent. The detection agent may comprise a ferrocene, a phenazine methosulfate, a vitamin (e.g., riboflavin), an enzyme (e.g., horseradish peroxidase) or a derivative thereof. In some embodiments, a detection agent is a ferrocene or a derivative thereof. For example, a detection agent may have one of the following chemical formulas:

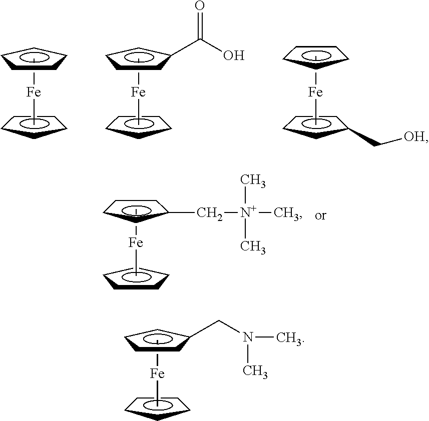

In some embodiments, a fluid comprises a detection agent that is a ferrocene carboxylic acid, a hydroxymethylferrocene, a (ferrocenylmethyl)dimethylamine, or a derivative thereof. For example, a detection agent may be a ferrocene monocarboxylic acid. Some ferrocene carboxylic acids may have a particularly advantageous association constant (Kₐ) with a cucurbit[7]uril trapping agent in aqueous fluids.

A fluid described herein may comprise a detection agent at a variety of suitable concentrations. In some embodiments, a fluid comprises a detection agent at a concentration of greater than or equal to 0.2 mM, greater than or equal to 0.3 mM, greater than or equal to 0.4 mM, greater than or equal to 0.5 mM, greater than or equal to 0.6 mM, greater than or equal to 0.7 mM, greater than or equal to 0.8 mM, greater than or equal to 0.9 mM, greater than or equal to 1 mM, greater than or equal to 1.2 mM, greater than or equal to 1.4 mM, greater than or equal to 1.6 mM, greater than or equal to 1.8 mM, greater than or equal to 2 mM, greater than or equal to 3 mM, greater than or equal to 4 mM, greater than or equal to 5 mM, greater than or equal to 6 mM, greater than or equal to 7 mM, greater than or equal to 8 mM, or greater than or equal to 9 mM. In some embodiments, a fluid comprises a detection agent at a concentration of less than or equal to 10 mM, less than or equal to 9 mM, less than or equal to 8 mM, less than or equal to 7 mM, less than or equal to 6 mM, less than or equal to 5 mM, less than or equal to 4 mM, less than or equal to 3 mM, less than or equal to 2 mM, less than or equal to 1.8 mM, less than or equal to 1.6 mM, less than or equal to 1.4 mM, less than or equal to 1.2 mM, less than or equal to 1 mM, less than or equal to 0.9 mM, less than or equal to 0.8 mM, less than or equal to 0.7 mM, less than or equal to 0.6 mM, less than or equal to 0.5 mM, less than or equal to 0.4 mM, less than or equal to 0.3 mM, or less than or equal to 0.2 mM. Combinations of these ranges are also possible (e.g., greater than or equal to 0.1 mM and less than or equal to 10 mM, greater than or equal to 0.1 mM and less than or equal to 2 mM, or greater than or equal to 0.2 mM and less than or equal to 2 mM). Other ranges are also possible. In some embodiments, a fluid does not comprise a detection agent (e.g., a kit may comprise one fluid comprising a detection agent and another lacking the detection agent).

Some fluids described herein comprise an indicator. An indicator may be configured to produce a detectable signal as a result of an interaction with a detection agent. As described above, in some embodiments, an interaction between an indicator and a detection agent is a chemical reaction. An indicator and a detection agent may interact directly, or may interact with the involvement of additional chemical species (e.g., one or more auxiliary agents) as described in greater detail below.

Any of a variety of appropriate indicators may be used. In some embodiments, an indicator comprises an aromatic portion. For example, an indicator may comprise alternating single and double bonds between carbon atoms. In some embodiments, an indicator is a chromogenic substrate for peroxidase activity. An indicator may be benzidine or a benzidine derivative, such as 3,3′,5,5′-tetramethylbenzidine (TMB). In some embodiments, an indicator is a 2,2′-Azinobis [3-ethylbenzothiazoline-6-sulfonic acid]-diammonium salt (ABTS), an o-phenylenediamine (OPD), a 7-Hydroxy-3H-phenoxazin-3-one 10-oxide (Resazurin), or a suitable derivative thereof. Further non-limiting examples of indicators include 5-Amino-2,3-dihydrophthalazine-1,4-dione (Luminol) and 10-Acetyl-3,7-dihydroxyphenoxazine (Amplex Red).

A fluid may comprise an indicator having any of a variety of suitable concentrations. In some embodiments, a fluid comprises an indicator having a concentration of greater than or equal to 1 mM, greater than or equal to 2 mM, greater than or equal to 5 mM, greater than or equal to 10 mM, greater than or equal to 25 mM, greater than or equal to 50 mM, greater than or equal to 75 mM, greater than or equal to 100 mM, greater than or equal to 125 mM, greater than or equal to 150 mM, greater than or equal to 175 mM, greater than or equal to 200 mM, or greater than or equal to 225 mM. In some embodiments, a fluid comprises an indicator having a concentration of less than or equal to 250 mM, less than or equal to 225 mM, less than or equal to 200 mM, less than or equal to 175 mM, less than or equal to 150 mM, less than or equal to 125 mM, less than or equal to 100 mM, less than or equal to 75 mM, less than or equal to 50 mM, less than or equal to 25 mM, less than or equal to 10 mM, less than or equal to 5 mM, or less than or equal to 2 mM. Combinations of these ranges are also possible (e.g., greater than or equal to 1 mM and less than or equal to 250 mM, greater than or equal to 10 mM and less than or equal to 100 mM, or greater than or equal to 25 mM and less than or equal to 75 mM). Other ranges are also possible. In some embodiments, a fluid does not comprise an indicator (e.g., a kit may comprise one fluid comprising an indicator and another lacking the indicator).

An interaction between an indicator and a detection agent may produce a detectable signal and/or result in the production of a species that generates a detectable signal. This may comprise the production of a state change in the indicator that creates a detectable signal. For example, an indicator may change color (e.g., change the color(s) of light that it absorbs), fluoresce, exhibit luminescence (e.g., chemiluminescence), and/or produce an electrical signal upon interaction with a detection agent. It is also possible for an indicator to stop absorbing light (e.g., of one or more colors), stop fluorescing, stop exhibiting luminescence and/or chemiluminescence, and/or stop producing an electrical signal upon interaction with a detection agents. Such changes may also be considered to be detectable signals. In some embodiments, a fluid comprises an indicator that is a colorimetric indicator. A colorimetric indicator may exhibit a visible color change upon interaction with a detection agent. The color change may allow the presence of the analyte to be determined by visual inspection of a fluid.

In some embodiments, an interaction between an indicator and a detection agent is a chemical reaction. The chemical reaction may be irreversible or reversible. In some embodiments, the chemical reaction is a redox reaction. For example, a detection agent and an indicator may each act as a redox reagent. In some embodiments, a fluid and/or plurality of fluids comprises an indicator that is an electron acceptor and a detection agent is an electron donor. It is also possible for a detection agent to be a catalyst for a separate reaction of an indicator. Non-limiting examples of further possible interactions between a detection agent and an indicator include chelation, hydrolysis reactions, and interactions that cause a change in the pH of a fluid wherein the interaction takes place. In some embodiments, the pH of a fluid may be buffered such that pH changes due to the interaction between a detection agent and an indicator are mitigated.

In some embodiments, an interaction between an indicator and a detection agent is mediated by one or more auxiliary agents. In some embodiments, an auxiliary agent is a chemical species that catalyzes or participates in a reaction between a detection agent and an indicator. For example, an auxiliary agent may be a redox reagent or a redox catalyst. In some embodiments, an auxiliary agent is included in one or more fluids described above. For example, an auxiliary agent may be included in a fluid comprising a trapping agent a detection agent, a fluid comprising an indicator, and/or a fluid comprising a trapping agent, a detection agent, and an indicator. It is also possible for an auxiliary agent to be provided in a separate fluid that does not comprise an indicator, a trapping agent, or a detection agent.

In some embodiments, an auxiliary agent is a peroxide, such as hydrogen peroxide.

A fluid described herein may have any of a variety of suitable pH values. In some embodiments, a fluid has a pH of greater than or equal to 2, greater than or equal to 3, greater than or equal to 4, greater than or equal to 5, greater than or equal to 6, greater than or equal to 7, greater than or equal to 8, or greater than or equal to 9. In some embodiments, a fluid has a pH of less than or equal to 10, less than or equal to 9, less than or equal to 8, less than or equal to 7, less than or equal to 6, less than or equal to 5, less than or equal to 4, or less than or equal to 3. Combinations of these ranges are also possible (e.g., greater than or equal to 2 and less than or equal to 10, greater than or equal to 3 and less than or equal to 9, greater than or equal to 4 and less than or equal to 8, greater than or equal to 3 and less than or equal to 5, or greater than or equal to 7 and less than or equal to 9). Other ranges are also possible.

In some embodiments, it may be advantageous for a fluid comprising a detection agent to have a pH of greater than or equal to 8, since, without wishing to be bound by any particular theory, relatively high pH values may improve the solubility of some detection agents by converting a carboxylic acid moiety present in a detection agent to a carboxylate moiety.

In some embodiments, it is advantageous for a fluid comprising a detection agent to have a pH of greater than or equal to 3 and less than or equal to 5 because it may be desirable to perform an assay at a pH in this range. This pH ranges may accelerate a reaction between some indicators and detection agents.

A fluid described herein may be buffered to any appropriate pH value, as described above. Any of a variety of suitable buffer systems may be used. In some embodiments, a fluid comprises a tris buffer, an acetate buffer, and/or a citrate buffer.

According to some embodiments, a method comprises detecting a signal. For example, a method may comprise detecting the presence or absence of a detectable signal (e.g., a color change, as illustrated in FIG. 3) that arises due to interaction of a detection agent with an indicator. In some embodiments, the signal arises from the indicator (e.g., after interaction with the detection agent) and/or in a fluid comprising the indicator. The presence or absence of a detectable signal may be used to determine whether a target analyte is present in a substance. For example, as discussed above, in some embodiments, in the presence of a target analyte, a detection agent may interact with an indicator to produce a detectable signal (e.g., a color change of the indicator), whereas in the absence of the analyte, the detection agent will not produce the detectable signal.

A detectable signal may be an optical signal (e.g., a change in absorbance, transmittance, fluorescence, or luminescence). In some embodiments, a detectable signal is an electrical or electrochemical signal. An indicator may produce a plurality detectable signals. For example, in some embodiments, a color change of an indicator is associated with a change in an absorbance and a change in a transmittance of a fluid in which the indicator is suspended, dispersed, and/or dissolved. In some embodiments, a color change is a change from a clear solution in the absence of an analyte to a colored solution in the presence of the analyte. The change from a clear solution to a colored solution may be associated with an increase in absorbance and a decrease in transmittance at a wavelength of visible light. It should be noted that is also possible for a change from a colored solution in the absence of an analyte to a clear solution in the presence of the analyte to occur, and that such a change may be associated with a decrease in absorbance and an increase in transmittance at a wavelength of visible light. In some embodiments, an external light source may be provided to assist with the observation of an optical signal and/or must be provided in order to observe an optical signal. For instance, detecting a fluorescence or luminescence signal may comprise and/or require exposing the fluid to external light of a suitable wavelength for observing fluorescence or luminescence. For example, the light source may provide UV light.

Any of a variety of appropriate methods may be used to detect a signal produced by an indicator interaction. For example, a signal may be detected by visual inspection, spectrometry (e.g., UV/VIS spectrometry), fluorimetry, or using a camera (e.g., a cell phone camera). A detectable signal may be used to determine the presence or absence of an analyte. In some embodiments, a detectable signal may be used to quantify an amount or concentration of an analyte (e.g., by comparison with a reference standard).

In the absence of the target analyte, the detectable signal may be absent, have an intensity that is not detectable by eye, and/or have an intensity that is within the range of the noise associated with a signal having zero intensity.

A signal produced by a process herein may reach steady state over any of a variety of appropriate timescales. In some embodiments, the signal may reach steady state relatively rapidly, which may be advantageous for field-detection of target analytes. In some embodiments, a signal reaches steady state in a period of less than or equal to 60 minutes, less than or equal to 50 minutes, less than or equal to 40 minutes, less than or equal to 30 minutes, less than or equal to 25 minutes, less than or equal to 20 minutes, less than or equal to 15 minutes, less than or equal to 10 minutes, less than or equal to 9 minutes, less than or equal to 8 minutes, less than or equal to 7 minutes, less than or equal to 6 minutes, less than or equal to 5 minutes, less than or equal to 4 minutes, less than or equal to 3 minutes, less than or equal to 2 minutes, less than or equal to 1 minute, less than or equal to 50 seconds, less than or equal to 40 seconds, less than or equal to 30 seconds, less than or equal to 20 seconds, or less than or equal to 10 seconds. In some embodiments, a signal reaches steady state in a period of greater than or equal to 20 seconds, greater than or equal to 30 seconds, greater than or equal to 40 seconds, greater than or equal to 50 seconds, greater than or equal to 1 minute, greater than or equal to 2 minutes, greater than or equal to 3 minutes, greater than or equal to 4 minutes, greater than or equal to 5 minutes, greater than or equal to 6 minutes, greater than or equal to 7 minutes, greater than or equal to 8 minutes, greater than or equal to 9 minutes, greater than or equal to 10 minutes, greater than or equal to 15 minutes, greater than or equal to 20 minutes, greater than or equal to 25 minutes, greater than or equal to 30 minutes, greater than or equal to 40 minutes, or greater than or equal to 50 minutes. Combinations of these ranges are also possible (e.g., greater than or equal to 10 seconds and less than or equal to 60 minutes, greater than or equal to 30 seconds and less than or equal to 30 minutes, or greater than or equal to 1 minutes and less than or equal to 20 minutes). Other ranges are also possible.

The compositions, kits, and methods described herein may be suitable for targeted detection of any of a variety of appropriate target analytes. A target analyte may be a controlled substance. For example, an analyte may be a street drug. An analyte may be an opioid such as fentanyl, or an amphetamine, such as methamphetamine. In some embodiments, an analyte has at least one of the chemical structures:

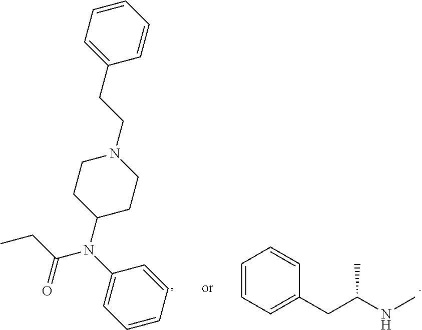 [SMILES: CCC(=O)N(c1ccccc1)C1CCN(CCc2ccccc2)CC1 ; CN[C@@H](C)Cc1ccccc1]

In some embodiments a target analyte is a pharmaceutical composition. For example, a target analyte may be a cancer medication. In some embodiments, a target analyte is a toxin or pesticide. In some embodiments, a target analyte is amantadine.

A composition, kit, or method described herein may be used to detect a target analyte having any of a variety of suitable masses. In some embodiments, a target analyte has a mass of greater than or equal to 5 micrograms, greater than or equal to 10 micrograms, greater than or equal to 25 micrograms, greater than or equal to 50 micrograms, greater than or equal to 75 micrograms greater than or equal to 100 micrograms, greater than or equal to 250 micrograms, greater than or equal to 500 micrograms, or greater than or equal to 750 micrograms. There is no particular upper limit to a mass of an analyte that can be detected, but in some embodiments, a target analyte having a mass of less than or equal to 1000 micrograms, less than or equal to 750 micrograms, less than or equal to 500 micrograms, less than or equal to 250 micrograms, less than or equal to 100 micrograms, less than or equal to 75 micrograms, less than or equal to 50 micrograms, less than or equal to 25 micrograms, or less than or equal to 10 micrograms is detected. Combinations of these ranges are also possible (e.g., greater than or equal to 5 micrograms and less than or equal to 1000 micrograms, greater than or equal to 10 micrograms and less than or equal to 1000 micrograms, or greater than or equal to 75 micrograms and less than or equal to 1000 micrograms). Other ranges are also possible.

A target analyte described herein may be detected in a fluid in any of a variety of concentrations. In some embodiments, an analyte may have a concentration in the fluid in which it is detected of greater than or equal to 0.5 mM, greater than or equal to 0.75 mM, greater than or equal to 1 mM, greater than or equal to 2.5 mM, greater than or equal to 5 mM, greater than or equal to 7.5 mM, greater than or equal to 10 mM, greater than or equal to 15 mM, greater than or equal to 20 mM, greater than or equal to 30 mM, greater than or equal to 50 mM, or greater than or equal to 100 mM. There is no particular upper limit to the concentration of an analyte that may be detected, but in some embodiments, an analyte may have a concentration in the fluid in which it is detected of less than or equal to 1 M, less than or equal to 100 mM, less than or equal to 50 mM, less than or equal to 30 mM, less than or equal to 25 mM, less than or equal to 20 mM, less than or equal to 15 mM, less than or equal to 10 mM, less than or equal to 7.5 mM, less than or equal to 5 mM, less than or equal to 2.5 mM, less than or equal to 1 mM, or less than or equal to 0.75 mM. Combinations of these ranges are also possible (e.g., greater than or equal to 0.5 mM and less than or equal to 30 mM, greater than or equal to 1 mM and less than or equal to 30 mM, or greater than or equal to 5 mM and less than or equal to 30 mM). Other ranges are also possible.

In some embodiments, a target analyte is detected when pure (e.g., pure fentanyl or pure methamphetamine). However, in some embodiments a composition, kit, or method described herein may be used to determine the presence of a target analyte in a substance. A substance may be an unknown substance (i.e., a substance of unknown chemical composition) or may be a substance with known composition (e.g., which may be used as a control). Any of a variety of appropriate substances may be analyzed. For example, a substance may be a powder, a residue, or a fluid. In some embodiments, a substance is sprayed with one or more fluids such that if an analyte is present in the substance, at least a portion of the analyte is able to join the one or more fluids, thereby producing a detectable signal.

The following examples are intended to illustrate certain embodiments of the present disclosure, but do not exemplify the full scope of the disclosure.

### Example 1

This example demonstrates the use of an exemplary trapping agent, cucurbit[7]uril (CB[7]); an exemplary detection agent, ferrocene monocarboxylic acid (FCA); and an exemplary indicator 3,3′,5,5′-tetramethylbenzidine (TMB) in an aqueous solution for the detection of the analyte amantadine (AM).

In this experiment, CB[7] hydrate, 99+% was purchased from Strem Chemicals. Ferrocene monocarboxylic acid was purchased from MilliporeSigma. Glacial acetic acid was purchased from Sigma-Aldrich and sodium acetate trihydrate was purchased from Fisher Chemical. The glacial acetic acid and sodium acetate trihydrate were employed to prepare a 0.2 M acetate buffer (pH 4). Hydrogen peroxide (H₂O₂) was purchased from CVS pharmacy and 3,3′,5,5′-tetramethylbenzidine (TMB) was purchased from Alfa Aesar. Dimethylformamide (DMF) was purchased from Sigma Aldrich. Amantadine HCl was purchased from Sigma-Aldrich.

A test solution was prepared according to the following procedure: 20 microliters of 1.5 mM FCA in tris buffer, pH 8.5, and 20 microliters of 16 mM CB[7] were added into 270 μL of 0.2 M acetate buffer solution, pH 4.0, and incubated for 2 minutes. The effective concentrations of each of the reagents was approximately 0.1 mM FCA and 1.0 mM CB[7]. After the short incubation, 5 μL of 3% hydrogen peroxide and 5 μL of 60 mM TMB were added to the solution.

A sample of unaltered test solution served as a control solution. A displacement solution was prepared identically to the test solution, except that 100 mM AM was introduced to act as an analyte.

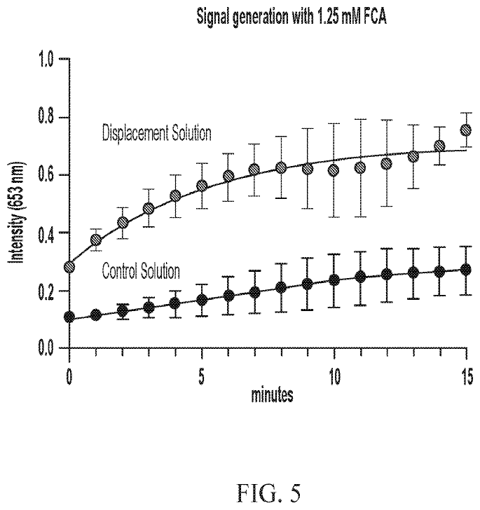

Real-time absorption measurements were obtained using a Varioskan LUX multimode microplate reader (ThermoFisher Scientific). For absorption measurements, a clear 96-well flat bottom plate was used to prepare solutions for quantitative analysis of signal development. Both complexes were spectroscopically characterized in acetate buffer using the microplate reader. FIG. 5 presents absorption intensity of the control solution and the displacement solution. As observed in FIG. 5, the absorbance signal of the control solution signal is significantly lower than that of the displacement solution signal, demonstrating a color change in the presence of AM.

These results demonstrate the ability of the compositions described herein to act as assays for target analytes.

### Example 2

This example demonstrates the use of an exemplary trapping agent, cucurbit[7]uril (CB[7]); an exemplary detection agent, ferrocene monocarboxylic acid; and an exemplary indicator 3,3′,5,5′-tetramethylbenzidine (TMB) in an aqueous solution for the detection of the analyte fentanyl.

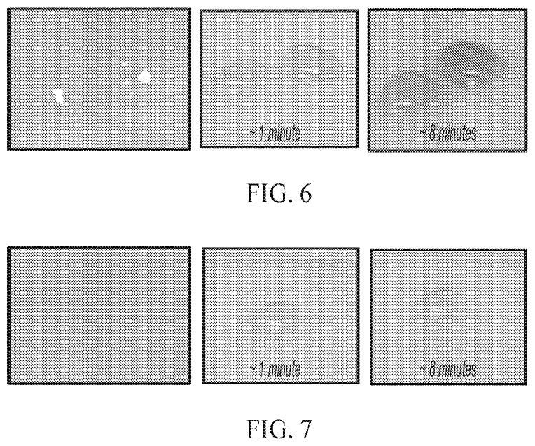

In this example, a solution comprising 16 mM CB[7] and 1.5 mM FCA in an acetate buffer was prepared as described in Example 1. The solution was added dropwise onto two grains of fentanyl powder. As a control, the solution was also added dropwise to a surface that had not been exposed to fentanyl. FIG. 6 presents the color evolution of the solution contacting the fentanyl powder. As shown, the drops contacting the fentanyl powder gradually became darker and more absorbent, indicating the successful detection of fentanyl. Over the same time period, the control solution demonstrated no visible color change. This is shown in FIG. 7.

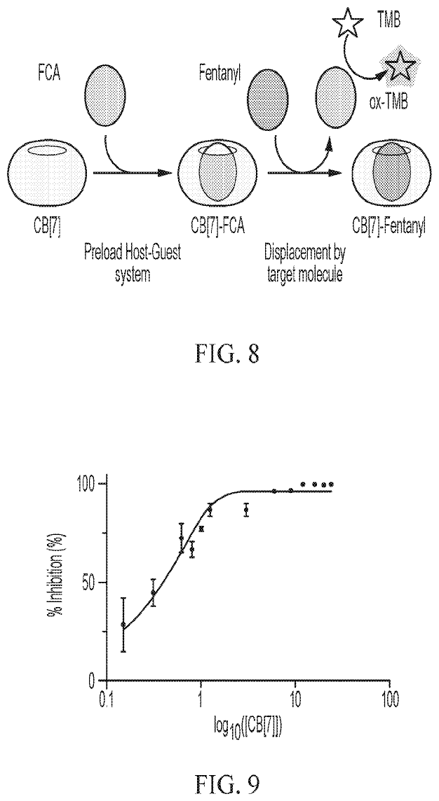

FIG. 8 schematically depicts some of the interactions and reactions that are believed to occur during the preparation of the solution comprising CB[7] and FCA and the subsequent addition of this solution to fentanyl powder. As can be seen in this Figure, upon mixing the FCA and CB[7] solutions, FCA from the FCA solution associates with CB[7] from the CB[7] solution. The addition of this mixed solution fentanyl allows the fentanyl to interact with CB[7]. This interaction causes association of fentanyl with CB[7], which displaces FCA from its association with CB[7]. This displacement frees the FCA to interact with TMB, thereby oxidizing TMB.

These results demonstrate the ability of the compositions described herein to act as an assay for fentanyl.

### Example 3

This example describes a process for determining an appropriate concentration of an exemplary trapping agent (cucurbit[7]uril (CB[7])) in an aqueous solution for detecting an analyte. The aqueous solution comprising the exemplary trapping agent further included an exemplary detection agent (ferrocene monocarboxylic acid (FCA)), and an exemplary indicator (3,3′,5,5′-tetramethylbenzidine (TMB)).

Test solutions including differing amounts of CB[7] were prepared in a manner similar to that described in Example 1. The differences from the test solutions described in Example 1 were as follows: 10 microliters of 1.25 mM FCA in tris buffer was employed instead of 20 microliters of 1.5 mM FCA in tris buffer; 10 microliters of the CB[7] solutions having varying CB[7] concentrations were employed instead of 20 microliters of 16 mM CB[7]; 135 μL of 0.2 M acetate buffer solution was employed instead of 270 μL of 0.2 M acetate buffer solution; 2.5 μL of 3% hydrogen peroxide was employed instead of 5 μL of 3% hydrogen peroxide; and 2.5 μL of 60 mM TMB was employed instead of 5 μL of 60 mM TMB.

Absorption measurements were performed on the test solutions as described in Example 1 after 15 minutes of incubation. These absorption measurements were employed to determine a percentage inhibition of the interaction of FCA with TMB by the following procedure: absorption measurements were performed on four replicates of each test solution; these four absorption measurements were averaged to determine an averaged absorption measurement (AVG) for each test solution; the minimum (MIN) and maximum (MAX) of the averaged absorption measurements were determined; and the percent inhibition for each sample was calculated by the following formula:

$$Percent Inhibition ( test solution ) = ( 1 - \frac{( AVG ( test solution ) - MIN )}{M A X - MIN} ) \cdot 100 \% .$$

FIG. 9 shows the percentage inhibition of this interaction as a function of CB[7] concentration. As can be seen from FIG. 9, the percentage inhibition of this interaction plateaus at a CB[7] concentration of approximately 12 mM. Accordingly, and without wishing to be bound by any particular theory, it is believed that concentrations of CB[7] close to 12 mM may be particularly beneficial for detecting the presence of target analytes. It is believed that such concentrations are high enough to prevent appreciable background signal while low enough to allow for quantitative displacement of the detection agent therefrom upon exposure to a target analyte.

### Example 4

This example demonstrates the use of an exemplary trapping agent, cucurbit[7]uril (CB[7]); an exemplary detection agent, ferrocene monocarboxylic acid (FCA); and an exemplary indicator 3,3′,5,5′-tetramethylbenzidine (TMB) in an aqueous solution for the detection of the analyte amantadine (AM) at various concentrations.

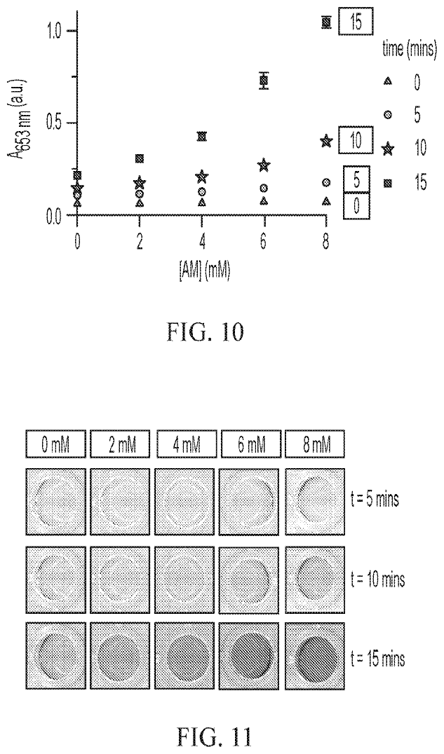

A test solution was prepared as described in Example 3, except that the concentration of CB[7] in the CB[7] solution was 12 mM. Displacement solutions were prepared identically to the test solution, except that varying concentrations of AM were introduced to act as an analyte. These concentrations were 2 mM, 4 mM, 6 mM, and 8 mM. Real-time absorption measurements were obtained as described in Example 1. FIG. 10 shows the results of these real-time absorption measurements plotted graphically for the test solution and different displacement solutions. FIG. 11 shows photographs of the test solution and the displacement solutions at various time points.

These results demonstrate the ability of the compositions described herein to act as assays for target analytes at various concentrations.

### Example 5

This example demonstrates the use of an exemplary aqueous solution for the detection of the analyte fentanyl upon addition of solid fentanyl thereto. The exemplary aqueous solution comprised an exemplary trapping agent, cucurbit[7]uril (CB[7]); an exemplary detection agent, ferrocene monocarboxylic acid; and an exemplary indicator 3,3′,5,5′-tetramethylbenzidine (TMB).

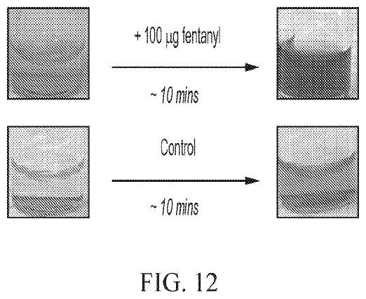

The exemplary aqueous solution was prepared in the same manner that the solution was prepared in Example 2. 100 micrograms of fentanyl was added to one sample of the exemplary aqueous solution. This sample was photographed upon fentanyl addition and at 10 minutes after fentanyl addition. An otherwise-equivalent exemplary aqueous solution to which fentanyl was not added was also photographed at these time points. These photographs are shown in FIG. 12. As can be seen from FIG. 12, the sample to which the fentanyl was added exhibited a much higher degree of color change than the sample to which the fentanyl was not added, thereby demonstrating the ability of the exemplary aqueous solution to act as an assay for fentanyl.

### Example 6

This example describes various exemplary indicators and detection agents for detecting fentanyl.

FCA test solutions were prepared in the same manner as the test solutions described in Example 1 except that the concentration of CB[7] was 12 mM and various other indicators were present instead of TMB. HRP test solutions were prepared in this same manner except that they also included horseradish peroxidase (HRP) as a detection agent instead of FCA. Both types of test solutions were added to centrifuge tubes containing fentanyl and to empty centrifuge tubes. Photographs were taken of each centrifuge tube after several minutes.

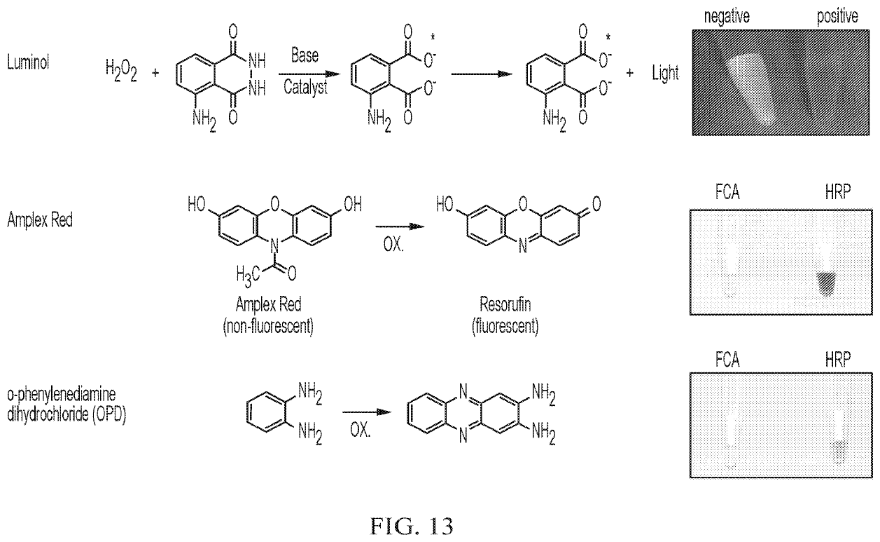

FIG. 13 shows the various indicators that were employed and photographs of selected centrifuge tubes. These indicators include 5-Amino-2,3-dihydrophthalazine-1,4-dione (Luminol), 10-Acetyl-3,7-dihydroxyphenoxazine (Amplex Red), and o-phenylenediamine dihydrochloride (OPD). As can be seen from FIG. 13, the test solution including luminol fluoresced in the absence of fentanyl but did not fluoresce in the presence of fentanyl. This is believed to indicate that the presence of free FCA suppressed the reaction between hydrogen peroxide to generate fluorescent species that would otherwise have occurred. As can also be seen in FIG. 13, the free FCA was not effective for oxidizing Amplex Red or ODP in the presence of hydrogen peroxide. However, free HRP was suitable for this purpose. Thus, it is believed that HRP is a suitable detection agent for use Amplex Red and ODP indicators.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” is an abbreviation of weight percentage. As used herein, “at %” is an abbreviation of atomic percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition for detecting the presence of a target analyte, comprising:
a fluid in which a detection agent, an indicator, and a trapping agent are dissolved, suspended, and/or dispersed, wherein:
at least a portion of the detection agent is associated with the trapping agent,
interaction of the detection agent with the indicator in the presence of the fluid yields a detectable signal,
the detectable signal is absent, has an intensity that is not detectable by eye, and/or has an intensity that is within the range of the noise associated with a signal having zero intensity, and
the target analyte has a higher affinity for the trapping agent than the detection agent in the presence of the fluid.

2. A kit for detecting the presence of a target analyte, comprising:
a first fluid in which a detection agent and a trapping agent are dissolved, suspended, and/or dispersed; and
a second fluid in which an indicator is dissolved, suspended, and/or dispersed;
wherein:
at least a portion of the detection agent is associated with the trapping agent,
interaction of the detection agent with the indicator in the presence of the first fluid and the second fluid yields a detectable signal, and
the target analyte has a higher affinity for the trapping agent than the detection agent in the presence of the first fluid and the second fluid.

3. A method of detecting a target analyte, the method comprising:
exposing a substance to a fluid and/or a plurality of fluids;
detecting the presence or absence of a detectable signal; and
determining whether a target analyte is present in the substance based on the presence or absence of the detectable signal, wherein:
prior to the exposure of the substance to the fluid and/or the plurality of fluids, a detection agent and a trapping agent are dissolved, suspended, and/or dispersed in the fluid and/or in a first fluid in the plurality of fluids,
prior to the exposure of the substance to the fluid and/or the plurality of fluids, at least a portion of the detection agent in the fluid and/or the first fluid is associated with the trapping agent,
prior to the exposure of the substance to the fluid and/or the plurality of fluids, an indicator is dissolved, suspended, and/or dispersed in the fluid and/or in a second fluid in the plurality of fluids,
in the absence of the target analyte, the detectable signal is absent, has an intensity that is not detectable by eye, and/or has an intensity that is within the range of the noise associated with a signal having zero intensity,
the target analyte has a higher affinity for the trapping agent than the detection agent in the presence of the fluid and/or the plurality of fluids, and
the detectable signal arises due to interaction of the detection agent with the indicator in the presence of the fluid and/or the plurality of fluids.

4. The method of any preceding claim, wherein the substance is exposed to a single fluid.

5. The method of any preceding claim, wherein the plurality of fluids comprises a first fluid in which the detection agent and the trapping agent are dissolved, suspended, and/or dispersed and a second fluid in which the indicator is dissolved, suspended, and/or dispersed

6. The method of any preceding claim, wherein exposing the substance to the fluids and/or the plurality of fluids comprises spraying the fluid and/or the plurality of fluids onto the substance

7. The method of any preceding claim, wherein the fluid, first fluid, second fluid, and/or plurality of fluids are aqueous.

8. The composition, kit, or method of any preceding claim, wherein the analyte is a controlled substance

9. The composition, kit, or method of any preceding claim, wherein the analyte is an street drug

10. The composition, kit, or method of any preceding claim, wherein the analyte is an opioid.

11. The composition, kit, or method of any preceding claim, wherein the analyte is fentanyl.

12. The composition, kit, or method of any preceding claim, wherein the association between the trapping agent and the detection agent blocks and/or reduces the accessibility of the detection agent to the indicator.

13. The composition, kit, or method of any preceding claim, wherein the association between the trapping agent and the detection agent is a guest/host interaction.

14. The composition, kit, or method of any preceding claim, wherein exposure of the target analyte to the fluid, the first fluid and and/or the plurality of fluids causes the target analyte to associate with the trapping agent, thereby displacing the detection agent from its association with the target analyte.

15. The composition, kit, or method of any preceding claim, wherein the trapping agent is a macrocycle.

16. The composition, kit, or method of any preceding claim, wherein the trapping agent is a cucurbituril.

17. The composition, kit, or method of any preceding claim, wherein the trapping agent is cucurbit[7]uril or a derivative thereof.

18. The composition, kit, or method of any preceding claim, wherein the indicator is a colorometric indicator.

19. The composition, kit, or method of any preceding claim, wherein the indicator is an electron acceptor.

20. The composition, kit, or method of any preceding claim, wherein the indicator is a benzidine or a benzidine derivative.

21. The composition, kit, or method of any preceding claim, wherein the indicator is 3,3′,5,5′-tetramethylbenzidine (TMB).

22. The composition, kit, or method of any preceding claim, wherein the interaction between the detection agent and the indicator is a redox reaction.

23. The composition, kit, or method of any preceding claim, wherein the detection agent is an electron donor.

24. The composition, kit, or method of any preceding claim, wherein the interaction between the detection agent and the indicator is a reaction catalyzed by the detection agent.

25. The composition, kit, or method of any preceding claim, wherein the interaction between the detection agent and the indicator comprises chelation.

26. The composition, kit, or method of any preceding claim, wherein the interaction between the detection agent and the indicator is a hydrolysis reaction.

27. The composition, kit, or method of any preceding claim, wherein the interaction between the detection agent and the indicator causes a pH of the fluid and/or the plurality of fluids to change.

28. The composition, kit, or method of any preceding claim, wherein the detection agent is a ferrocene carboxylic acid.

29. The composition, kit, or method of any preceding claim, wherein the detection agent is a ferrocene monocarboxylic acid.

30. The composition, kit, or method of any preceding claim, wherein the detectable signal is an optical signal.

31. The composition, kit, or method of any preceding claim, wherein the detectable signal is an absorbance.

32. The composition, kit, or method of any preceding claim, wherein the detectable signal is a transmittance.

33. The composition, kit, or method of any preceding claim, wherein the detectable signal is fluorescence.

34. The composition, kit, or method of any preceding claim, wherein the detectable signal is chemiluminescence.

35. The composition, kit, or method of any preceding claim, wherein the Kₐ between the trapping agent and the target analyte is greater than or equal to 5×10⁶ the Kₐ between the trapping agent and the detection agent is less than or equal to 1.3×10⁶ and.

36. The composition, kit, or method of any preceding claim, wherein the ratio of the Kₐ between the trapping agent and the target analyte and the Kₐ between the trapping agent and the detection agent is greater than or equal to 10.

37. The composition, kit, or method of any preceding claim, wherein fluid or the plurality of fluids further comprises an auxiliary agent.

38. The composition, kit, or method of any preceding claim, wherein the auxiliary agent is a peroxide.

39. The composition, kit, or method of any preceding claim, wherein the auxiliary agent is hydrogen peroxide.

40. The composition, kit, or method of any preceding claim, wherein the fluid or of at least one of the plurality of fluids is buffered.

41. The composition, kit, or method of any preceding claim, wherein the pH of the fluid or of at least one of the plurality of fluids is between 3 and 9.
